(19) United States
(12) Patent Application Publication
(10) Pub. No.: US 2017/0160251 A1
(43) Pub. Date: Jun. 8, 2017

(54) Aircraft Fuel Tank Inerting System Adapted To Compute The Quantity Of Oxygen Present In An Inerting Gas Injected Into Said Tank

(71) Applicant: Zodiac Aerotechnics, Roche la Moliere (FR)

(72) Inventors: Bruno Reynard, Francheville (FR); Frédéric Denat, Saint Just Saint Rambert (FR)

(21) Appl. No.: 15/367,926

(22) Filed: Dec. 2, 2016

(30) Foreign Application Priority Data
Dec. 3, 2015 (FR) 1561818

(51) Int. Cl. G01N 33/00; G01M 15/04; G01N 21/64; B64D 37/32

(52) U.S. Cl. CPC G01N 33/0057; B64D 37/32; G01M 15/042; G01N 21/643; G01N 21/645

(57) ABSTRACT

An inerting system for inerting a fuel tank of an aircraft. The system includes: a sensor including a phosphorescent material, arranged in a distribution mechanism for distributing an inerting gas and in contact with the inerting gas; a light source illuminating the phosphorescent material; a measurement mechanism for measuring the phosphorescence of the phosphorescent material; and a computation mechanism for computing the quantity of oxygen present in the inerting gas as a function of the attenuation of the phosphorescence as measured that is directly related to the quantity of oxygen in the inerting gas.

### FIELD OF THE INVENTION

The present invention relates to the technical field of inerting systems for a fuel tank of an aircraft, such as an airplane, a helicopter, or the like, and it relates more particularly to an inerting system adapted to compute the quantity of oxygen present in an inerting gas injected into a fuel tank.

### BACKGROUND OF THE INVENTION

In the field of aviation and aircraft construction, it is well known that inerting systems can be used to generate nitrogen, or some other inert gas such as, for example, carbon dioxide, and to inject it into the fuel tanks for safety reasons in order to reduce the risk of such tanks exploding.

Such inerting systems are also known as “On-Board Inert Gas Generation Systems” or “OBIGGSs”.

A conventional inerting system in the prior art includes, in general, an OBIGGS fed with air, e.g. with bleed air diverted from at least one engine. The model using bleed air diverted from at least one engine is currently the OBIGGS model that is in most widespread use. In such a system, the bleed air is generally diverted from one or more engines from the “intermediate pressure” opening and/or from the “high pressure” opening depending on the flight situation. It should be noted that it is advantageous to use bleed air for the air conditioning because bleed air has a relatively high pressure, and a relatively high temperature, so that the air can be adjusted over a wide range of desired pressures and temperatures. The OBIGGS is coupled to the fuel tank of the aircraft and it separates the oxygen from the air.

The OBIGGS is generally constituted by an air separation module that, for example, contains zeolite membranes through which a flow of air is pressed. Due to the different mass transfer rates of nitrogen and of oxygen, the system splits the flow of air so that a high nitrogen content air flow and a high oxygen content air flow are obtained. The nitrogen-enriched air fraction (nitrogen being considered to be the inerting gas) is fed to the fuel tanks so that the mixture of air and of aviation fuel vapor present there is shifted and removed from the tanks. The oxygen-enriched air fraction can be fed back into the passenger cabin after having been treated with suitable means and/or into the combustion chambers of the jet engines in order to improve combustion. The devices necessary for this operation, such as compressors, filters, air and water cooling modules, and the like are incorporated into the inerting gas installation.

Thus, when the ratio between the fuel and oxygen, in the empty portion of the tank is less than the flammability limit defined in compliance with the requirements of the Federal Aviation Administration (FAA) as given in detail in Document AC25.981-2A dated Sep. 19, 2008 and entitled “FUEL TANK FLAMMABILITY REDUCTION MEANS” and its Appendices, no spontaneous ignition can take place. In the above, making a fuel tank inert consists in particular in keeping the oxygen content present in said tank below a certain threshold, and in particular below 12%.

In the above, it is therefore important to know precisely what quantity of oxygen is present in the gas that is discharged by said air separation module and that is to be injected into the fuel tank(s) of the aircraft.

For that purpose, a known inerting system generally incorporates a device for measuring the quantity of oxygen, which device is arranged in an inerting gas feed duct for feeding the inerting gas to the fuel tank, that duct being situated downstream from the separation means.

In the state of the art, an inerting system is known that includes a measurement device having a casing defining a chamber through which the inerting gas can flow. That casing encloses measurement means provided with a zirconia oxygen probe or sensor for taking the necessary measurements in said gas to determine the oxygen concentration. The zirconia probe is powered, in particular by a fixed voltage, and operates at a temperature that is relatively hot.

The drawback with that type of inerting system is that it incorporates means for measuring the quantity of oxygen that operate hot and that are based on a metal material. The inerting system is thus voluminous and heavy, and it implements a relatively complex system of connectors.

In addition, that type of measurement device is sensitive to environmental conditions, and the measurement given by it can drift in uncontrolled manner. The measurements taken by the zirconia probe vary as a function of the environmental conditions under which said probe is used, and in particular as a function of the temperature and pressure at which the measurement means are kept.

Finally, the measurement taken by the probe drifts in a manner that is random over time due to the ageing of the sensitive element based on zirconia.

### SUMMARY OF THE INVENTION

An object of the invention is thus to remedy those drawbacks by proposing an inerting system for inerting a fuel tank of an aircraft that, in addition, makes it possible to compute the quantity of oxygen present in an inerting gas injected into the fuel tank, in a manner that is simple, reliable, and accurate over time, without having any significant impact on the weight of said system.

Another object of the invention is, in particular, to provide such an inerting system in which the measurement of the quantity of oxygen is less sensitive to environmental conditions, so as to limit the drift in its measurement, or indeed so as to remove said drift.

To this end, the invention provides an inerting system for inerting at least one fuel tank of an aircraft, such as, for example, an airplane or a helicopter, or the like, said inerting system comprising:
at least one inerting gas generator fed with bleed air diverted from at least one engine and/or with air from a passenger cabin; and
distribution means for distributing inerting gas to the fuel tank(s), connected to the inerting gas generator and incorporating a measurement device for measuring the quantity of oxygen present in said inerting gas.

In accordance with the invention, the measurement device comprises:
a sensor including a phosphorescent material, arranged in the distribution means and in contact with the inerting gas;
a light source illuminating the phosphorescent material;
measurement means for measuring the phosphorescence of the phosphorescent material; and
computation means for computing the quantity of oxygen present in the inerting gas as a function of the attenuation of the phosphorescence as measured that is directly related to the quantity of oxygen in the inerting gas.

Certain materials have the property of emitting photons while they are going from an excited state to a lower-energy level. The excited state is obtained, for example, by absorption of electromagnetic radiation emitted by the light source. This results in an effect of attenuating or extinguishing the phosphorescence, this effect being related to the presence of a molecule in the reaction medium, e.g. to the presence of oxygen. The more the phosphorescence of the material is attenuated rapidly, the more the quantity of oxygen present in the inerting gas surrounding the phosphorescent material is high. As a function of the time constant of the attenuation of the phosphorescence of a material, the computation means are suitable for computing the quantity of oxygen present in the gas. In practice, the computation means determine the variation in the intensity of the phosphorescence as a function of time, and compute a decay constant. It is this decay constant that makes it possible to compute the quantity of oxygen present in the gas as a function of the phosphorescent material used.

Thus, the inerting system of the invention is simpler to implement because it does not necessarily require any chamber specific to the measurement, and does not create any hot spot in the system. The measurement device of the inerting system operates cold and in real time. In addition, it is more compact, lighter in weight, and less complex than the above-described prior art systems.

Determining the quantity of oxygen present in the inerting gas at the outlet of the inerting gas generator makes it possible to perform a diagnostic assessment of said generator in order to check its state of operation, and its performance, and in order to decide whether or not replacement of or maintenance on said generator should be scheduled.

In a particular embodiment of the inerting system, the sensor is connected via optical fibers firstly to the light source and secondly to the measurement means, said light source and said measurement means being arranged outside the distribution means for distributing the inerting gas.

In this way, the light source is remote from the phosphorescent material, and illuminates the phosphorescent material by means of the optical fibers. The same applies for the measurement means that may also be remote because they observe the phosphorescence of the material via the optical fibers. Optical fibers are flexible, light in weight, and can be bent, and this considerably facilitates assembling the system. The system is made neither more voluminous nor heavier.

In a particular embodiment, the phosphorescent material comprises a polymer matrix and a phosphorescent compound, and, for example, is in the form of a pellet.

Advantageously, the light source comprises at least one light-emitting diode (LED).

When the phosphorescent material used also has fluorescence in response to the illumination by the light source, and in order to make the measurement as accurate as possible, the sensor includes means for removing fluorescent emissions and reflection of the incident light coming from the light source off said phosphorescent material.

The measurement means are of any suitable type, and, for example, include a photo-detector.

### BRIEF DESCRIPTION OF THE DRAWINGS

Other advantages and characteristics appear more clearly from the following description of the measurement device of the invention, given way of non-limiting example and with reference to the accompanying drawings, in which:

FIG. 1 is a block diagram showing the principle of an inerting system of the invention;

FIG. 2 is a diagrammatic view showing how the measurement device is arranged relative to the means for distributing the inerting gas;

FIG. 3 is a diagrammatic view of an embodiment of the measurement device of the inerting system of the invention; and

FIG. 4 is a graph showing, for a determined phosphorescent material, the time constant of the attenuation of the phosphorescence of said phosphorescent material when it is illuminated by the light source, as a function of the quantity of oxygen present in the inerting gas.

### DETAILED DESCRIPTION OF THE INVENTION

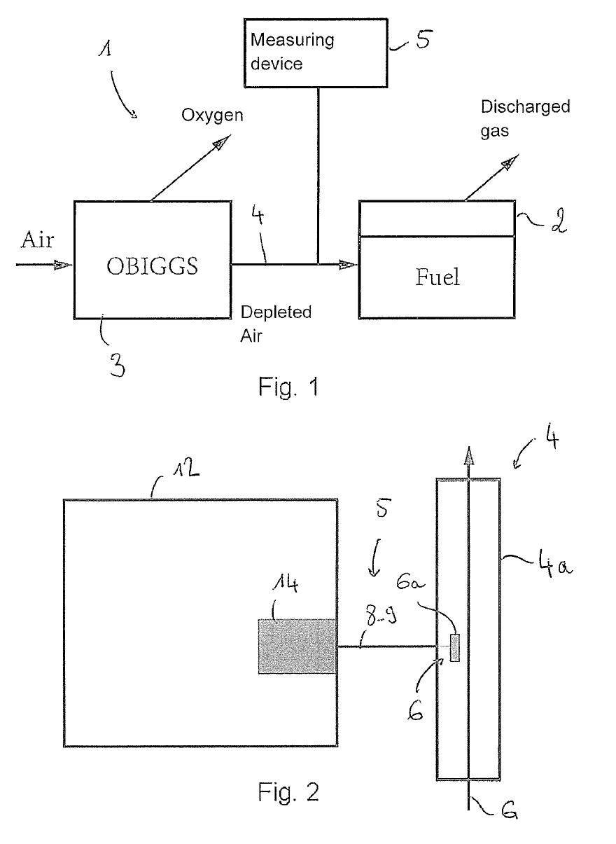

With reference to FIG. 1, the invention relates to an inerting system (1) for inerting one or more fuel tanks (2) of an aircraft, which inerting system generates nitrogen, or some other inert gas such as, for example, carbon dioxide, and injects it into said fuel tank(s) (2) for safety reasons in order to reduce the risk of said tanks (2) exploding.

The inerting system (1) generally comprises an On-Board Inert Gas Generation System (OBIGGS) (3) fed with air, e.g. bleed air diverted from at least one engine and/or air coming from a passenger cabin of the aircraft. The inerting gas generator (3) is, for example, in the form of zeolite membranes through which air is pressed in such a manner as to obtain firstly an inerting gas having a high nitrogen content, and secondly a gas having a high oxygen content.

The inerting system (1) further comprises distribution means (4) such as ducts for example, for distributing the inerting gas to the fuel tank(s) (2), which ducts are connected to the inerting gas generator (3). The purpose of the injected inerting gas is to make the fuel tank(s) (2) inert, i.e. to make it possible to reduce the oxygen content present in said tank(s) (2), and in particular to keep that content under a certain threshold, and preferably less than 12%.

In accordance with the invention, the inerting system (1) further comprises a measurement device (5) for measuring the quantity of oxygen present in the inerting gas injected into the tank(s) (2).

More precisely, and with reference to FIG. 2, the measurement device (5) includes a sensor (6) containing a phosphorescent material (7). The sensor (6) is arranged inside the distribution means (4), such as inside a gas duct (4a) or inside a valve, downstream from the inerting gas generator (3) and upstream from the tanks (2), and such that the phosphorescent material (7) is in contact with the inerting gas. The arrow (G) passing through the duct (4a) represents the inerting gas flowing through it.

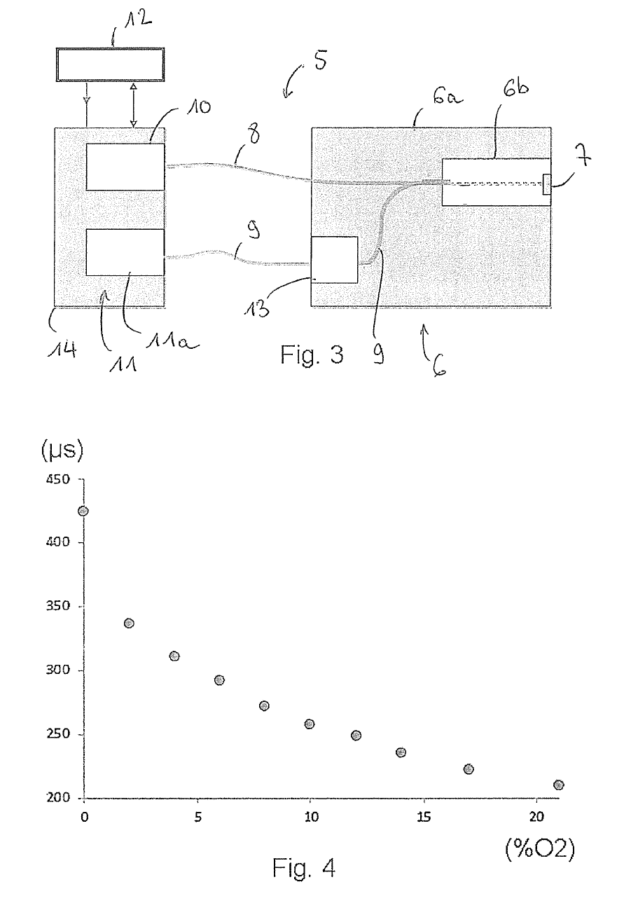

With reference to FIG. 3, and in a particular embodiment, the sensor (6) is in the form of a probe (6a) immersed in the distribution means (4) and in contact with the inerting gas. This probe (6a) incorporates an adapter (6b) to one end of which the phosphorescent material (7) is fastened. The adapter (6b) is itself positioned at one end of the probe (6a) so as to put the phosphorescent material (7) into contact with the inerting gas.

Another end of the adapter (6b) is provided with first and second optical fibers (8, 9), arranged in alignment with said phosphorescent material (7), i.e. having their ends positioned in register with said phosphorescent material (7). The first optical fiber(s) (8) are connected to a light source (10) comprising at least one light-emitting diode for illuminating said phosphorescent material (7). The second optical fiber(s) (9) are connected to measurement means (11) for measuring the phosphorescence of the phosphorescent material (7) that is, for example, in the form of a photo-detector (11a).

In this way, the light source (10) illuminates the phosphorescent material (7) for a lapse of time in the range 0.1 milliseconds (ms) to a few ms, e.g. 3 ms, so as to cause said phosphorescent material (7) to go into an excited state. After the phosphorescent material (7) has been illuminated and while said phosphorescent material (7) is going to a lower energy level, said phosphorescent material emits photons, thereby revealing its phosphorescence. The phosphorescence is measured by the photo-detector (11a) by means of the second optical fibers (9). The attenuation of the phosphorescence as measured by the photo-detector (11a), which attenuation is directly related to the quantity of oxygen in the inerting gas, is computed by computation means (12).

The computation means (12) incorporate processing means suitable firstly for removing fluorescent emissions when the phosphorescent material (7) used is also fluorescent, and secondly for removing the reflection of the incident light from the light source (10) off said phosphorescent material (7). In another particular embodiment, the photo-detector (11a) observes the phosphorescence of the phosphorescent material (7) through an optical filter (13) suitable for performing these removal functions.

Preferably, the photo-detector (11a) and the light source (10) are part of a module (14) connected to the computation means (12), such as a computer, making it possible firstly to power the module (14) electrically and secondly to compute the quantity of oxygen in the inerting gas as a function of the data on the intensity of the phosphorescence that is received from the photo-detector (11a). The computation means (12) determine the variation in the intensity of the phosphorescence as a function of time, and compute a decay constant. It is this decay constant that makes it possible to compute the quantity of oxygen present in the gas as a function of the phosphorescent material (7) used.

As regards the phosphorescent material (7), various compositions are possible. The essential requirement lies in the fact that said material should go from a phosphorescent excited state, when it is illuminated by the light source (10), to a non-phosphorescent unexcited normal state.

In practice, the photo-detector (11a) measures said phosphorescence and the computation means (12) make it possible to compute the attenuation of the phosphorescence, namely the time constant of the attenuation of the phosphorescence, which attenuation depends, in particular, on the quantity of oxygen present in the inerting gas.

In this way, it is possible to generate, previously, a graph, such as the graph shown in FIG. 4, identifying the time constant of the attenuation of the phosphorescence of a phosphorescent material (7) determined in the presence of a known quantity of oxygen. The phosphorescent material (7) determined is characterized by a certain chemical composition and by a certain thickness.

In this way, by means of said graph, it is possible to determine the quantity of oxygen present in the inerting gas as a function of the time constant of the attenuation of the phosphorescence as computed by the computation means (12).

For the use under consideration, consisting in keeping the oxygen content present in the inerting gas injected into a fuel tank (2) under a certain threshold, in particular less than 12%, the phosphorescent material (7) must have significant attenuation of its phosphorescence in the presence of such a quantity of oxygen. Thus, it is preferable to have a phosphorescent material (7) having a large variation in its speed of attenuation of its phosphorescence in the presence of from 5% of oxygen in the gas to 15% of oxygen in the gas, and preferably in the presence of from 8% of oxygen to 12% of oxygen. The attenuation should be precise and fast, in particular within a time limit lying in the range 1 minute to 5 minutes, in view of the use related to aircraft safety.

In addition, the quantity of oxygen in the gas, which quantity is a function of the attenuation of the phosphorescence of a phosphorescent material (7), also depends on the temperature and on the partial pressure of oxygen present in the gas. Thus, the computation means (12) are also coupled to means making it possible to measure the temperature and/or the partial pressure of oxygen in order to enable the quantity of oxygen to be computed.

In the preferred embodiment, compatible with the use in question as regards the speed of attenuation of the phosphorescence, namely the response time of the material and the range of the quantity of oxygen over which the attenuation is most significant, the phosphorescent material (7) used is, for example, in the form of a pellet of diameter in the range a few millimeters (mm) to about 1 cm and of thickness of 150 micrometers (μm). The pellet comprises a polymer matrix including a phosphorescent compound. The phosphorescent compound may be a complex of porphyrin of a group-10 metal, such as a complex of porphyrin of palladium or of platinum.

By way of example, the phosphorescent compound may be:
5,10,15,20-Tetrakis(2,3,4,5,6-pentafluorophenyl)porphyrin-Pd(II):

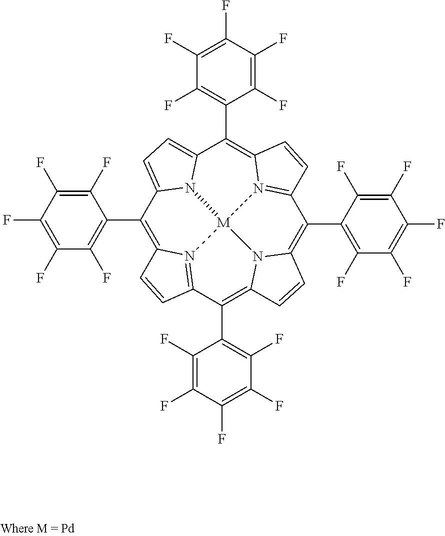

2,3,7,8,12,13,17,18-Octaethylporphyrin-Pd(II) or 2,3,7,8,12,13,17,18-Octaethylporphyrin-Pt(II):

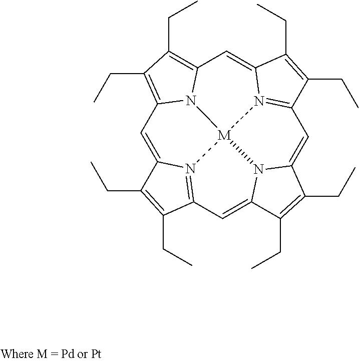

Meso-Tetraphenylporphyrin-Pd(II) or Meso-Tetraphenylporphyrin-Pt(II):

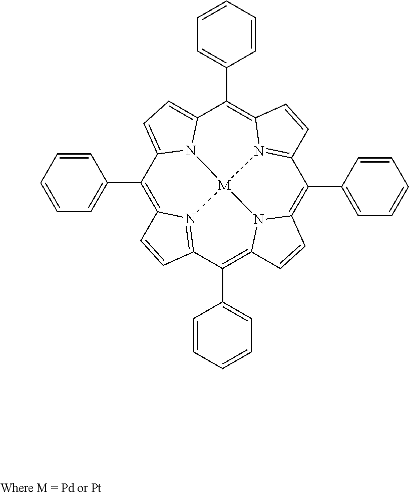

5,10,15,20-Tetrakis(4-hydroxymethylphenyl)porphyrin-Pt(II):

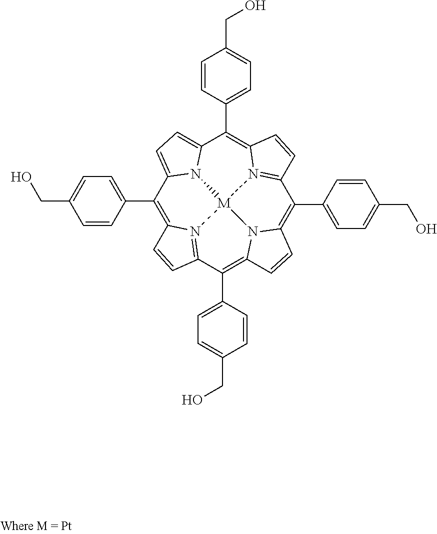

Other compounds may be used without going beyond the ambit of the invention, such as, in particular, organic compounds of the type:

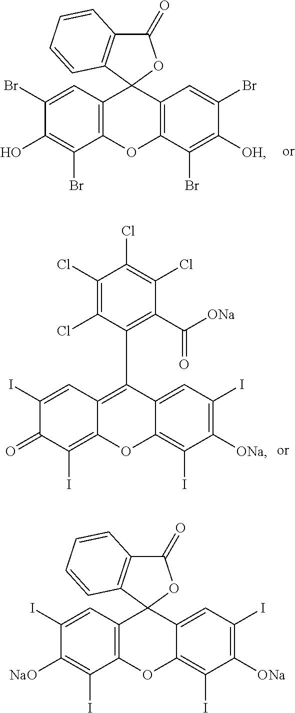

The polymer matrix is mixed with the phosphorescent compound. The polymer matrix needs to be chosen as a function of the use being considered. In other words, the polymer matrix must have a certain amount of resistance to aviation fuel vapors and be bonded to the phosphorescent compound. Ideally, it is possible to have one or more covalent bonds between the molecules of the matrix and the phosphorescent compound.

The polymer matrix may be polyurethane obtained by the reaction of a compound having at least two isocyanate functions and a compound having at least two alcohol functions.

By way of example, the polymer matrix may be a polyurethane obtained by the reaction between:
a PTDI (poly(propylene glycol), toluene 2, 4-diisocyanate-terminated):

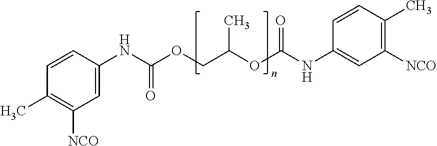

And
a TMP (trimethylopropane):

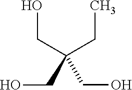

A surfactant may also be added to the mixture.

In another embodiment, the polymer matrix may be a polyurethane obtained by the reaction between:
a TMP (trimethylopropane):

And
a PHD (Poly(hexamethylene diisocyanate):

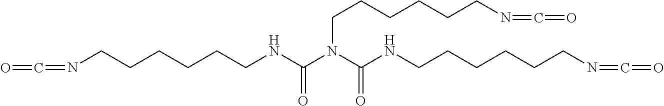 [SMILES: O=C=NCCCCCCNC(=O)N(CCCCCCN=C=O)C(=O)NCCCCCCN=C=O]

A phosphorescent compound is then incorporated into the polyurethane to form the phosphorescent material (7) of the present invention.

As appears from the above, the invention provides an inerting system (1) for inerting a fuel tank of an aircraft, which system is capable of computing the quantity of oxygen present in the inerting gas injected into the fuel tank(s) (2). The quantity of oxygen is computed simply reliably, and accurately over time, without having any significant impact on the weight of said system because it implements lightweight optical fibers. The system implements measurement by luminescence that does not involve any rise in temperature for the measurement device (5). Measurement is performed cold, without any risk for the inerting system (1) and the aircraft. The phosphorescence of the material is not degradable or sensitive to environmental conditions, so that the measurement does not drift over time.

Computing the quantity of oxygen then makes it possible to act on the inerting gas generator (3) as a function of the computed value so as to generate an inerting gas that has a higher or lower oxygen content for injecting into the fuel tank(s) (2).

Finally, the invention makes it possible to determine the quality of the inerting gas at the outlet of the inerting gas generator, and thus makes it possible to perform a diagnostic assessment of said generator in order to check its state of operation, and its performance, and in order to decide whether or not replacement of or maintenance on said generator should be scheduled.

1. An inerting system for inerting at least one fuel tank of an aircraft, said system comprising:
at least one inerting gas generator fed with bleed air diverted from at least one engine and/or with air from a passenger cabin;
distribution means for distributing inerting gas to the fuel tank(s), connected to the inerting gas generator and incorporating a measurement device for measuring a quantity of oxygen present in said inerting gas.
a sensor including a phosphorescent material, arranged in the distribution means and in contact with the inerting gas;
a light source illuminating the phosphorescent material;
measurement means for measuring phosphorescence of the phosphorescent material; and
computation means for computing the quantity of oxygen present in the inerting gas as a function of the attenuation of the phosphorescence as measured that is directly related to the quantity of oxygen in the inerting gas.

2. The inerting system according to claim 1, characterized in that the sensor is connected via optical fibers firstly to the light source and secondly to the measurement means, said light source and said measurement means being arranged outside the distribution means for distributing the inerting gas.

3. The inerting system according to claim 1, characterized in that the phosphorescent material comprises a polymer matrix and a phosphorescent compound.

4. The inerting system according to claim 1, characterized in that the light source comprises at least one light-emitting diode.

5. The inerting system according to claim 1, characterized in that the measurement device includes means for removing fluorescent emissions and a reflection of incident light coming from the light source off said phosphorescent material.

6. The inerting system according to claim 1, characterized in that the measurement means include a photo-detector.
